(19) United States
(12) Patent Application Publication
(10) Pub. No.: US 2026/0117195 A1
(43) Pub. Date: Apr. 30, 2026

(54) METHOD FOR CONVERTING XY HUMAN IPSC LINES INTO XX HUMAN IPSC LINES VIA INTERMEDIATE XO STAGE

(71) Applicant: Conception Biosciences, Inc., Berkeley, CA (US)

(72) Inventor: Pablo Ignacio HURTADO GONZALEZ, Berkeley, CA (US)

(73) Assignee: Conception Biosciences, Inc., Berkeley, CA (US)

(21) Appl. No.: 19/373,632

(22) Filed: Oct. 29, 2025

Related U.S. Application Data
(60) Provisional application No. 63/713,951, filed on Oct. 30, 2024.

(51) Int. Cl. C12N 5/074

(52) U.S. Cl. CPC C12N 5/0696

(57) ABSTRACT

Provided are methods for converting XY human PSC lines into XX human PSCs lines by passaging and identifying colonies with an intermediate XO chromosomal state, followed by further passaging to identify colonies with an XX chromosomal composition.

### CROSS-REFERENCE TO RELATED APPLICATIONS

This application claims priority to U.S. Provisional Application No. 63/713,951, filed Oct. 30, 2024, entitled “METHOD FOR CONVERTING XY HUMAN IPSC LINES INTO XX HUMAN IPSC LINES VIA INTERMEDIATE XO STAGE,” the contents of which are incorporated by reference in their entirety.

### FIELD

The present disclosure relates to stem cell biology and, more specifically, to a method of altering the chromosomal composition of human pluripotent stem cells (PSCs), e.g., induced pluripotent stem cells (iPSCs) or embryonic stem cells (ESCs) for applications in regenerative medicine, genetic research, and therapeutic development.

### BACKGROUND

Human iPSC lines with specific chromosomal configurations hold significant promise for advancing research in genetics, regenerative medicine, and therapeutic applications. Female (XX) iPSC lines, in particular, offer unique insights into these fields. While successful conversion of XY (male) mouse embryonic stem cells (ESCs) to XX has been documented, most notably by Murakami et al. (2022), who demonstrated a two-step process that generated functional XX ESCs capable of differentiating into oocytes and producing healthy offspring. This approach has proven less straightforward in human pluripotent stem cell cultures.

Natural Y chromosome loss and chromosomal stability during passaging are well-documented phenomena in mouse ESC cultures (Hirota et al., 2017), facilitating the transition through an XO intermediate to an XX chromosomal state. However, in human iPSCs, knowledge and success rates regarding natural Y chromosome loss and chromosomal stability are considerably lower. This invention addresses these challenges by providing a novel, systematic method for generating XX human iPSCs from XY lines, allowing for greater utility of XX iPSCs in human-focused research and potential therapeutic applications.

### SUMMARY

This invention provides a method for converting XY human PSC lines into XX human PSCs lines by passaging and identifying colonies with an intermediate XO chromosomal state, followed by further passaging to identify colonies with an XX chromosomal composition. Additionally, the invention allows for an optional enhancement of the process through cell surface markers or a fluorescent reporter system that increases fluorescence intensity with additional X chromosomes, aiding in the selection of XX PSCs.

### BRIEF DESCRIPTION OF THE DRAWINGS

FIG. 1A shows the original iPSC line with a 46, XY karyotype and FIG. 1B shows an example of a 45, XO.

FIG. 2A shows the starting iPSC line with a 45, XO karyotype and FIG. 2B shows an example of a 46, XX subclone.

FIG. 3 shows the fluorescence of iPSCs after each passage.

FIG. 4A and FIG. 4B show exemplary differentially expressed cell surface markers between XY, XO and XX.

### DETAILED DESCRIPTION

Provided are methods for altering the chromosomal composition of human pluripotent stem cells (PSCs). In some embodiments, the pluripotent stem cells are induced pluripotent stem cells (iPSCs). In some embodiments, the pluripotent stem cells are embryonic stem cells (ESCs).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHOD

Step 1: Conversion of XY to XO iPSC Lines

1. Passaging XY PSCs—Multiple rounds of PSC passaging are performed on the XY PSC lines. During this process, a natural loss of the Y chromosome may occur, yielding XO PSC colonies. This process can potentially be enhanced by culturing the PSCs in the presence of a molecule that increases the chances of chromosomal abnormalities.

2. Identification of XO Colonies—Individual colonies are analyzed for chromosomal composition. Colonies identified as XO, where the Y chromosome has been naturally lost, are selected for the next step.

### Step 2: Conversion of XO to XX PSC Lines

1. Passaging XO PSCs—The selected XO PSC lines undergo additional rounds of passaging. During this stage, spontaneous duplication of the X chromosome can result in XX PSC colonies. This process can potentially be enhanced by culturing the PSCs in the presence of a molecule that increases the chances of chromosomal abnormalities.

2. Identification of XX Colonies—Each colony is examined for chromosomal content. XX colonies are identified based on the duplication of the X chromosome and are selected for downstream applications.

### A. Optional Enrichment to Increase Efficiency Using Cell Surface Markers:

A potential enrichment strategy involves sorting PSC populations based on differential expression of specific cell surface markers unique to XY, XO, or XX cells. Certain markers may exhibit varied expression profiles depending on chromosomal composition, enabling an effective separation of XO cells during Step 1 or XX cells during Step 2. By identifying and targeting these cell surface markers with antibodies or other binding agents, this approach allows for precise enrichment of the desired chromosomal variant, facilitating a faster and more selective isolation of XO or XX PSC lines without reliance on intracellular or fluorescent markers.

### B. Optional Enrichment to Increase Efficiency Using a Fluorescent Reporters:

A fluorescent reporter can be introduced in either the XY or XO PSC line, where fluorescence intensity correlates with the number of X chromosomes. This system enables rapid enrichment of XX PSCs, as these will exhibit higher fluorescence compared to XO or XY lines. The increased fluorescence simplifies the sorting and identification process, allowing for more efficient and accurate isolation of XX lines.

### C. Readout Method:

Fluorescence in situ hybridization (FISH) is used as the primary and reliable method for verifying the sex chromosomal content of the PSC colonies. FISH analysis provides a direct readout of sex chromosome numbers, confirming the XY, XO, or XX status of the PSCs with high precision.

G-Banding and Karyotyping was used as a final readout to ensure sex chromosome numbers and rule out other chromosomal abnormalities. Other methods could be used to determine sex chromosome numbers, such as gene expression of SRY, ddPCR with specific chromosome probes or protein levels.

### II. EXEMPLARY EMBODIMENTS

Among the provided embodiments are:

1. A method for converting XY human PSC lines to XX human PSC lines through passaging to generate an XO intermediate.

2. The use of specific surface markers that are differentially expressed between XY, XO and XX human PSC lines to aid in the enrichment of XX human PSC lines by indicating the sex chromosome numbers.

3. The use of a fluorescent reporter to aid in the enrichment of XX human PSC lines by indicating the presence of additional X chromosomes.

4. A method of culturing XY or XO human PSC lines with a molecule that can induce chromosomal aberrations, such as microtubule assembly inhibitors, to increase the chances of obtaining XO or XX PSC lines.

### III. EXAMPLES

The examples discussed below are intended to be purely exemplary of the invention and should not be considered to limit the invention in any way. The examples are not intended to represent that the experiments below are all or the only experiments performed.

### Example: Methods and Results

Method to Culture Human iPSCs

Human XY iPSCs were cultured using standard techniques. Briefly, cells were grown on vitronectin-coated plates (Fisher Scientific) with mTeSR-Plus medium (Stemcell Technologies) at 37° C. in 5% CO₂. The medium was changed every other day, and cells were passaged onto new plates every 3-7 days, depending on confluency. For subcloning, individual colonies were transferred into 96-well plates, with a portion of each colony reserved for consequent analysis, such as qPCR for SRY or FISH analysis. Subclones suspected to be XO or XX were then expanded, banked, and further analyzed by G-banding karyotyping.

Obtaining XO iPSC Line from an XY iPSC Line

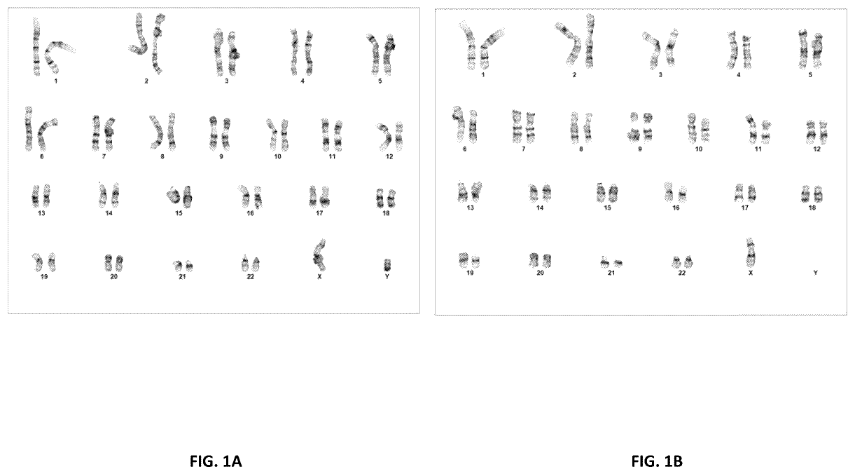

XY iPSCs were cultured following the methods described above. After several passages, some clones were identified with an XO karyotype, indicating the loss of the Y chromosome. FIG. 1A shows the original iPSC line with a 46, XY karyotype and FIG. 1B shows an example of a 45, XO subclone.

Obtaining XX iPSC Line from an XO iPSC Line

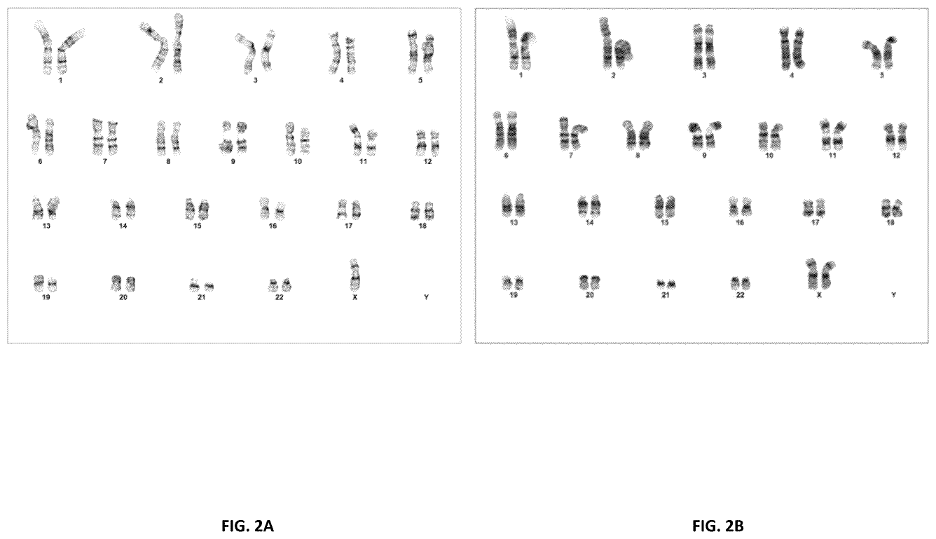

XO iPSCs were cultured following the methods described above. After several passages, some clones were identified with an XX karyotype, indicating the duplication of the X chromosome. FIG. 2A shows the starting iPSC line with a 45, XO karyotype and FIG. 2B shows an example of a 46, XX subclone.

Using Fluorescent Reporter to Enrich for XX iPSC Lines Vs XO iPSC Lines

Differential gene expression analysis between cells with different X chromosome copy numbers—1 X chromosome to 4 copies of X chromosome—revealed genes with expression levels influenced by X chromosome copy number (San Roman et al., 2023). One of these genes is KDM6A. An XO iPSC line with a fluorescent reporter tagged to KDM6A expression was generated using CRISPR/Cas9.

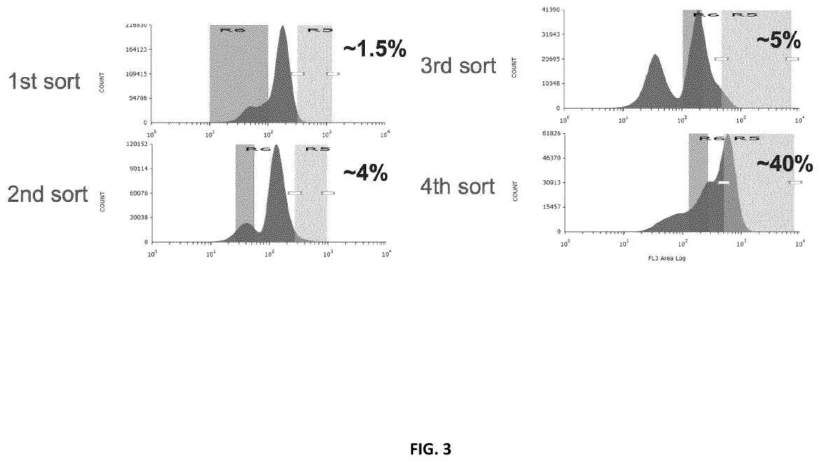

iPSCs were sorted at each passage based on increased fluorescence. By the fourth sort, approximately 40% of the iPSCs exhibited high fluorescence (FIG. 3). It was possible to isolate individual clones for testing by karyotyping to have XX chromosome composition.

List of Cell Surface Markers that are Differentially Expressed Between XY. XO and XX Lines

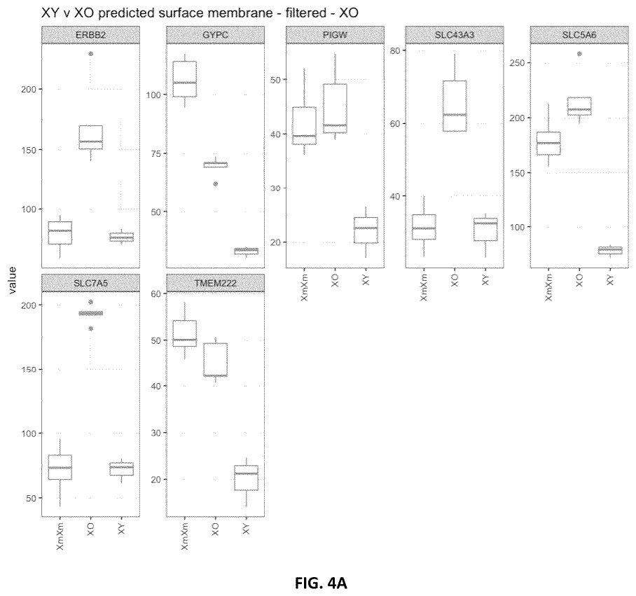

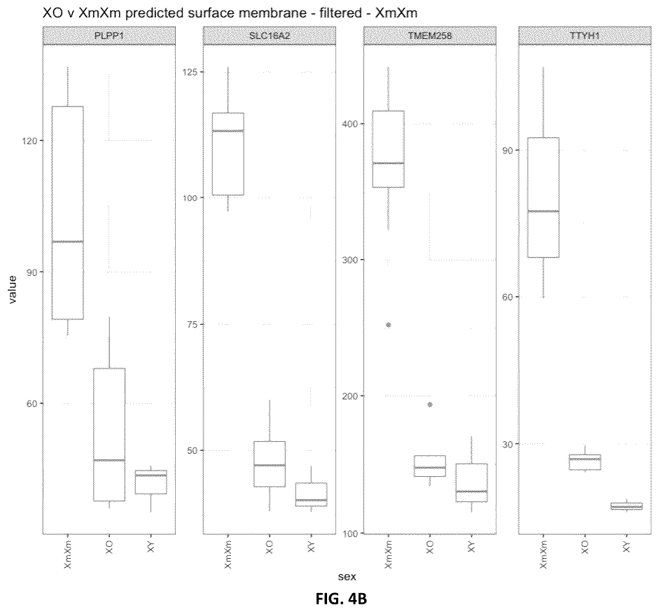

Differential gene expression analysis between XY, XO and XX iPSC lines, filtered by surface markers only, revealed genes with expression levels influenced by X chromosome copy number. By using antibodies targeting these specific genes, XY iPSC cultures can be enriched via sorting for the cells that are XO. Similarly, XO iPSC cultures can be enriched via sorting for the cells that are XX. Exemplary differentially expressed cell surface markers between XY, XO and XX are shown in FIG. 4A and FIG. 4B.

### CITATIONS

Hirota, T., Ohta, H., Mizutani, E., Miyauchi, H., Okuno, H., Matsuda, Y., Hosoi, Y., Nakata, A., Matsumoto, K., & Matsui, Y. (2017). Single male germ cells in vitro reconstitute spermatogenesis and produce offspring mice. Science, 357(6356), 232-235
Murakami, K., Gunesdogan, U., Zylicz, J. J., Tang, W. W. C., Sengupta, R., Kobayashi, T., Kim, S., Butler, R., Dietmann, S., & Hayashi, K. (2020). A stepwise differentiation model for XY male germ cell development in vitro. Nature, 582(7813), 593-598. https://doi.org/10.1038/s41586-020-2383-9
San Roman, A. K., Godfrey, A. K., Skaletsky, H., Bellott, D. W., Groff, A. F., Harris, H. L., Blanton, L. V., Hughes, J. F., Brown, L., Phou, S., Buscetta, A., Kruszka, P., Banks, N., Dutra, A., Pak, E., Lasutschinkow, P. C., Keen, C., Davis, S. M., Tartaglia, N. R., Samango-Sprouse, C., Muenke, M., & Page, D. C. (2023). The human inactive X chromosome modulates expression of the active X chromosome. Cell Genomics, 3(2), 100259. https://doi.org/10.1016/j.xgen.2023.100259

What is claimed is:

1. A method for converting XY human PSC lines to XX human PSC lines through passaging to generate an XO intermediate.

2. The use of specific surface markers that are differentially expressed between XY, XO and XX human PSC lines to aid in the enrichment of XX human PSC lines by indicating the sex chromosome numbers.

3. The use of a fluorescent reporter to aid in the enrichment of XX human PSC lines by indicating the presence of additional X chromosomes.

4. A method of culturing XY or XO human PSC lines with a molecule that can induce chromosomal aberrations, such as microtubule assembly inhibitors, to increase the chances of obtaining XO or XX PSC lines.
